SEQUENCE LISTING (1 sequence)

SEQ ID NO: 1 - DNA, 10042 bp, Artificial sequence
  Synthetic DT-A construct
ttcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagntctagagtcgaccaattctcatgtttgacagcttatcatcgcagatcctgagcttgtatggtgcactctcagtacaatctgctctgctgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgcccagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctgggtaccagctgctagtggatccaccggtgaagttcctatactttctagagaataggaacttcggaataggaacttctacgtagctaggccggtaccatggatcctcgacgctctcccttatgcgactcctgcattaggaagcagcccagtagtaggttgaggccgttgagcaccgccgccgcaaggaatggtgcatgcaaggagatggcgcccaacagtcccccggccacgggcctgccaccatacccacgccgaaacaagcgctcatgagctcctcgaggataacttgtttattgcagctttataatggttacaaataaagcaatagcatcacaaatttcacaaataaagcatttttttccatgcattctagttgtggtttgtccaaactcatcaatgtatcttatcatgtctgatcctcgaggagctcggtacccgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgacagaaatgaggtcatcaaatgtcttccaatgtgagattttgggccattctttatagcaaagattggataaggcgcatttttcttcaaagccttgttgtacgatctgactaagttatcttttaataattggtattcctgtttgttgcttgaagaattgccggtcctatttactcgttttaggactggttcagaattcctcaaaaattcatccaaatatacaagtggatcgatcctaccccttgcgctaaagaagtatatgtgcctactaacgcttgtctttgtctctgtcactaaacactggattattactcccagatacttattttggactaatttaaatgatttcggatcaacgttcttaatatcgctgaatcttccacaattgatgaaagtagctaggaagaggaattggtataaagtttttgtttttgtaaatctcgaggtatactcaaacgaatttagtattttctcagtgatctcccagatgctttcaccctcacttagaagtgctttaagcatttttttactgtggctatttcccttatctgcttcttccgatgattcgaactgtaattgcaaactacttacaatatcagtgatatcagattgatgtttttgtccattgtaaggaataattgtaaattcccaagcaggaattaatttctttaatgaggcttccagaattgttgctttttgcgtcttgtatttaaactggagtgatttgttgacaatatcgaaactcagcgaattgcttatgatagtattatagctcatgaatgtggctctcttgattgctgttccgttatgagtaatcatccaacataaataggttagttcagcagcacatgatgctattttttcccctgaaggtctttcaaacctttccacaaactgacgaaccaggaccttaggtggtgttttacataatatatcaaattgtggcatggtggaagcttgggctgcagttggacctgggagtggacacctgtggagagaaaggcaaagtggatgtcattgtcactcaagtgtatggccagatctcaagcctgccacacgtcaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagccggccctcgcagacagcgaattctagccatttaaatgcatgctgcagcccgggcgacgtcgacatttaaatgatcacggtaccgaattcgatatcaagcttggggctggggagcctcccccaggagccctataaaaccttcattccccaggactccgcccctgccctgctggcacccagaggctgaccaaggccctccccatgctgctggaggctggacaaccccctcccacacccagagctgtggaaggggagggagagctagcacttgctgttctgcaattactagatcaccctggatgcaccaggcccttgtagctcatggagacttcatctaggggacaaaggcagaggagacatgcccaggatgaaacagaaacagggggtgggtacgatccccgattcttcatacaaagcctcacgtgcctagatcctttgcactccaagacccagtgtgccctaagacaccagcactcaggagattgtgagactccctgatccctgcaccactctgagaccagaaactagaacttttattcctcatgctcctgaaatagatgtcttggcatttagtacattcttttccttgcactcccaacccagaatccagctccacagatacattgctactgtcatcataaaaagatcctgagcgctgccttattctgggtttggcagtggagtgctgccagacacagtcgatcgggacctagaaccttggttaggcataaagaagcaggatgtgatagaagaagtatttaatggtggaacgttgagactgtcctgcagacaagggtggaaggctctggctgaacagcgttgggaggcaattctccatggttctgtcacgtatctgtgtgtcttctgagcaaagacagcaacacctttttttttctggattgttgtttcaaggatgtttgtaaagcaggcatccttgcaagatgatatctctctcagatccaggcttgcttactgtcctagataataaagataatgtctcttacaacagatttgtttactgtcaaggacaatcgatacaatatgttcctccagagtaggtctgttttcaatccaagatcatgaagataatatcttcatcagagacaaaggctgagcaggtttgcaagttgtcccagtataagattgaggattcctaatctcaggtttctcaccagtggcacaaaccccttctgcacagcatccacctagactgctctggtcaccagagcagtctaggtggatgctgtgcagaaggggtttgtgccactggtgagaaacctgagattaggaatcctcaatcttatactgggacaacttgcaaacctgctcagcctttgtctctgatgaagatattatcttcatgatcttggattgaaaacagacctactctggaggaacatattgtatcgattgtccttgacagtaaacaaatctgttgtaagagacattatctttattatctaggacagtaagcaagcctggatctgagagagatatcatcttgcaaggatgcctgctttacaaacatccttgaaacaacaatccagaaaaaaaaaggtgttgctgtctttgctcagaagacacacagatacgtgacagaaccatggtaaccctacaagatttggggggggcaaggtgtactaatgtgaacatgaacctcatgctgtctgctaagctgtgagcagtaaaagcctttgcctctgactcaggagtctcatggactctgccagcattcacaaaactctggaaagttagcttatttgtttgcaagtcagtaaaatgtcagccccttcagagttactgacaaacaggtgggcactgagactgcactggccagctgggaatagagataggaggggacccagctggatgcagtgggcagtgggggtcatagagtcaagagggtacagaatacaatggggtcctagtaccatggtggaggtcagaaagagccctaaaagagagggtcaaggtaggaggttagtgaaggtccacctccaccctctccaggacagggacatcaggccacaattaatttctctgcagttggtgagtggtcatggtctctggagtccccagcatccagagtgtccctggtctagtggtcccccctttctgagccacagccactttctccatcaaatgaggccagtaatacccatcccatagtgatgctgtgaggatgagatgagcatctgtaagtgctgaagataatccctgacacatcccaagcattcagcagtgcaagcatacacttacacggcactccccagagccaggcatgtgctggtgcctcatacacgtgaccacatttgatcgtcacaatgaccctgtgagggagactgtgcaacagaggactgaccttgctcaaagacctcaggcgtttcccctcagagcctgagaggtcatctctttttttttttttttttcctttctttctttttcttttccatttctttttctttgcaagaggtcatctctaatgctttggaatatcctgccagattagagtccctttgttcacctgaaggtttgggccacaccagatagtctaacggtgtgatttgtgctgaaggttttgagccacactatatcagctagatttctagctcgagccttcgaagatctcctagggaagttcctatactttctagagaataggaacttcggaataggaacttcaccggtaccgagctcggatctgcgacctgcaggtcctcgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcgaatgccagatcgagacatagtgaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaacttgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctncaaatgtggtatggctgattatgatctgcggccgctcgagtctagcggccgctcgaggccggcaaggccggatcctcgactcgaagatccaataacttcgtatagcatacattatacgaagttataagtgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcggctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcactctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtata

(19) United States
(12) Patent Application Publication
(10) Pub. No.: US 2005/0215507 A1
(43) Pub. Date: Sep. 29, 2005

(54) Therapeutic anti-cancer DNA

(75) Inventors: Daniel Griffith Anderson, Framingham, MA (US); Robert Langer, Newton, MA (US); Janet Sawicki, Newton Square, PA (US); Weidan Peng, Haverford, PA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignees: Massachusetts Institute of Technology, Cambridge, MA (US); Lankenau Institute for Medical Research, Wynnewood, PA (US)

(21) Appl. No.: 11/074,323

(22) Filed: Mar. 4, 2005

Related U.S. Application Data
(60) Provisional application No. 60/550,912, filed on Mar. 4, 2004.

(51) Int. Cl. A61K 48/00; C07H 21/04; C12N 15/85

(52) U.S. Cl. 514/44; 435/455

(57) ABSTRACT

Aspects of the invention relate to nucleic acids molecules that are useful for specifically destroying selected cells, tissues, or organs. Aspects of the invention are useful for treating diseases (e.g., cancer) that affect specific cells, tissues, or organs.

### RELATED APPLICATIONS

This application claims priority under 35 U.S.C. § 119(e) from U.S. provisional patent application Ser. No. 60/550,912, filed Mar. 4, 2004, the entire disclosure of which is incorporated herein by reference.

### GOVERNMENT SUPPORT

This work was supported by NIH grant CA90841 (J.A.S.).

### FIELD OF THE INVENTION

The invention relates to expression vectors for killing cancer cells. The invention also relates to methods of treating cancer.

### BACKGROUND OF THE INVENTION

In 2004, it is estimated that 231,000 new cases of prostate cancer will be diagnosed in the United States and 29,000 men will die of advanced stage prostate cancer, making it the second leading cause of cancer deaths behind lung cancer. The high incidence of prostate cancer deaths is attributable to the fact that many patients have metastatic disease at the time of disease presentation. While surgery and radiation are often used successfully to treat the primary tumor, albeit often with significant unwanted side effects, there is currently no effective cure for metastatic prostate cancer. Hormone ablation therapy only effectively treats androgen-dependent tumors, while chemotherapy lacks tumor-specificity and administration of effective doses is limited by toxicity issues.

There is therefore a need in the art for methods and compositions for treating prostate cancer.

### SUMMARY OF THE INVENTION

The invention provides highly regulated nucleic acid constructs useful for killing cancer cells and treating patients with cancer. In particular, the invention provides, a highly regulated, prostate-specific, diphtheria toxin-encoding DNA. The invention also provides methods and compositions for delivering the nucleic acids constructs. In particular, nucleic acid constructs of the invention can be effectively delivered to cancer cells (e.g., to human prostate cancer cells) in combination with one or more poly(β-amino ester)s. In some embodiments, DNA delivery-effectively kills cells grown in culture as well as tumor cells in mice. The invention provides useful nanoparticles for the delivery of suicide (and therapeutic) genes for the treatment of cancer, including prostate cancer.

Aspects of the invention involve delivery of a nucleic acid to a cell to destroy the cell. In one embodiment, methods of the invention involve a toxin that is expressed only in a pre-selected cell type (e.g., a cancerous cell or a cell type that includes cancerous cells). For example, a construct of the invention may be targeted to certain prostate cells in order to kill prostate cancer cells (e.g., in the prostate, or cancer cells that have metastasized from the prostate).

In one aspect, the invention provides a nucleic acid construct including i) a first nucleic acid with a first promoter that is operably joined to a sequence encoding a recombinase, ii) a second nucleic acid with a second promoter, a sequence encoding a toxin, and at least one target sequence that is recognized by the recombinase. The second promoter and the sequence encoding the toxin are arranged such they are only operably joined if the recombinase acts on the at least one target sequence. Expression of the recombinase may be limited to a pre-selected cell type by using a first promoter that is selectively expressed in a certain cell type. Selective expression may be specific expression (e.g., expression is essentially limited to one or more specific cell types) or preferential expression (e.g., expression is relatively higher in one or more specific cell types). According to one aspect of the invention, the recombinase only acts on the target sequence(s) if the recombinase reaches a threshold level sufficient to promote the recombination event that leads to expression of the toxin. Therefore, even if low amounts of recombinase are expressed in a cell, the recombination event will not take place and the toxin encoding sequence will not be operably joined to the second promoter. In certain embodiments, there is little or no expression of the toxin if it is not operably joined to the second promoter. Therefore, the toxin is only expressed in cells where the level of recombinase reaches the threshold level. This allows for very tight control of toxin expression using a system where the recombinase is under control of a promoter that may be leaky (e.g., results in low levels of expression in certain tissues) provided that the selective expression of the recombinase results in above-threshold levels of the recombinase only in the selected cell or tissue type(s). In one embodiment, the recombinase promoter is a tissue specific promoter that is selectively active in non-vital tissue (e.g., a non-vital organ, for example, breast, thyroid, ovary, testes, or prostate).

According to aspects of the invention, each of the recombinase and toxin promoters may independently be modified promoters or may be associated with one or more transcription enhancer elements. For example, the recombinase promoter may be a prostate specific promoter such as a prostate specific antigen promoter or a modified version thereof (e.g., a PSA promoter, a modified PSA promoter, PSA-BC, etc.).

The recombinase promoter may be a tissue and/or cancer selective promoter. The toxin promoter (the second promoter described above) may be cell or tissue-selective, cancer selective, or cell/tissue/disease non-selective.

Each promoter independently may be a xenogeneic promoter (e.g., a promoter from a species other than the species of the subject or cells being targeted). Each promoter independently may be a mammalian promoter, a viral promoter, or a modified form thereof.

The toxin may be a natural protein or nucleic acid or a modified form thereof. The toxin may have enzymatic activity. The activity of the toxin may be directly toxic or it may be result in the production of another molecule that is toxic (e.g., by processing an endogenous or exogenously added compound to generate a toxic product). A toxin may be a diphtheria molecule (e.g., the A chain of diphtheria toxin).

According to aspects of the invention, the recombinase and toxin may be encoded on two separate nucleic acid molecules. Alternatively, the recombinase and toxin (and the promoters and recombinase target sequence(s)) may be on a single nucleic acid molecule (e.g., a linear, circular, single-stranded, or double stranded DNA, RNA, or other nucleic acid molecule such as a synthetic nucleic acid molecule).

According to aspects of the invention, nucleic acid constructs may be delivered virally or non-virally (e.g., in combination with a polymer such as a cationic polymer, for example, a C32 polymer described herein). Nucleic acid constructs may be administered to a subject in order to treat cancer (e.g., an epithelial cell cancer such as a prostate epithelial cell cancer) or other disease that affects a particular cell type (e.g., a benign hyperplasia or a benign tumor growth). Aspects of the invention may be used to treat metastatic cancer, particularly where metastatic cancer cells are of a particular cell type (a construct would be used with a recombinase that is under the control of a promoter that is selectively active in that cell type).

Effective treatments for metastatic cancer currently do not exist. Where other treatment modalities have failed, the potential of systemically-delivered gene therapy for the successful treatment of metastatic cancer is perhaps the biggest factor motivating research in this technically challenging field. The identification of tissue and/or tumor-specific promoter elements for targeting expression of therapeutic and toxic genes to tumor cells is useful for the practical application of systemically administered gene therapy (Shirakawa, T. K. et al., 1998, In vivo suppression of osteosarcoma pulmonary metastasis with intravenous osteocalcin promoter-based toxic gene therapy, Cancer Gene Ther., 5:274-280; Lee, 2002). Promoter elements may direct expression of therapeutic DNA to primary tumor and metastatic lesions. In one embodiment, there is little or no expression of therapeutic DNA in normal, non-cancerous cells. However, promoter elements may direct expression of a toxin to a particular tissue that contains both cancerous and non-cancerous tissue, particularly if the tissue is non-vital (e.g., non-vital organs or tissues) resulting in killing of the non-cancerous tissue in addition to the cancerous tissue. Accordingly, cancer specificity can be achieved by the destruction of all tissue affected by the cancer (i.e., both cancerous and non-cancerous cells in a non-vital organ or tissue). This may be therapeutically beneficial in order to get rid of cancer cells even if the treatment results in the loss of healthy tissue and/or a non-vital organ. For example, since the absence of normal prostate function is not life-threatening, the targeting therapeutic or toxic gene expression to normal prostate cells can be tolerated. Thus, compared to cancers in vital organs, gene therapy for the treatment of non-vital organ cancer, including metastatic cancers of non-vital organs (e.g., metastatic prostate cancer) is an especially attractive option.

According to aspects of the invention specific expression in non-vital organs can be used. A non-vital organ may be the prostate, spleen, a reproductive organ (e.g., the ovaries or testes), the breasts or the thyroid. The use of cell type selective targeting is particularly attractive for systemic delivery of a therapeutic nucleic acid. However, in certain embodiments, a therapeutic nucleic acid may be targeted to a particular organ or portion thereof by delivering it directly to the target site (e.g., by direct injection). In these embodiments, a nucleic acid that is less cell type selective may be used, because only the targeted region is exposed to the toxin.

As discussed above, aspects of the invention are useful to obtain very tightly regulated expression of a gene (e.g., the toxin gene) using a system of promoters, each of which may be leaky. While many promoters have been identified that have high activity in one cell-type as compared to others, these so-called “cell-specific” promoters are usually “leaky”, i.e., they are active at a low level in many different cell types. This minimal level of activity poses a problem in using these promoters to regulate the expression of genes encoding highly toxic proteins like DT-A (a single DT-A molecule is sufficient to kill a cell) (Yamaizumi, M. et al., 1978, One molecule of diphtheria toxin fragment A introduced into a cell can kill the cell, Cell, 15:245-250). However, these promoters may be used in a bipartite recombination system of the invention. For example, a bipartite Flp recombination system (Peng, W. et al., 2002, Regulated expression of diphtheria toxin in prostate cancer cells, Mol. Ther., 6:537-545) can be modified and used according to the invention to effectively restrict DT-A expression to specific cells (e.g., epithelial cells, cancer cells, etc.) in specific tissues or organs where promoter activity is above basal levels. Accordingly, cell type selective promoters can be used to target the expression of any toxin to a specific tissue using methods and compositions of the invention. For example, the prostate-specific promoter (PSA) can be used to target expression to the prostate. Other examples of tissue specific promoters include but are not limited to ovary mesothelin (MSLN), human epididymis protein 4 (HE4), secretory leukoprotease inhibitor (SLPI), ceruloplasmin, ovarian-specific promoter 1 (OSP-1), testis lactate dehydrogenase (LDHC), angiotensin-converting enzyme (ACE), thyroid-thyroglobulin, and breast ErbB2/HER2.

Aspects of the invention also address the low expression levels of may cell type selective promoters. By using a bipartite system, the activated toxin gene can be expressed by a strong promoter that is not necessarily cell type selective. However, cell-specific promoters that are useful for driving expression of a recombinase in therapeutic constructs of the invention are often weak promoters that activate low levels of transcription. Accordingly, a modified cell-specific promoter can be used to obtain threshold levels of recombinase in target cells. For example, a modified promoter/enhancer of the prostate specific antigen (PSA) gene, PSE-BC, can be used to regulate the expression of FLP recombinase. PSA is expressed normally by secretory cells in the prostate. It is also expressed by prostate tumor cells in androgen-dependent and androgen-independent tumors as well as in cells in androgen-independent metastatic foci. While native PSA enhancer and promoter elements allow for prostate-specific expression, the level of transcriptional activity driven by this promoter is low. PSE-BC augments gene expression approximately 19-fold above native levels while retaining tissue discriminatory capability, thus allowing for robust expression of Flp recombinase (Wu, L. et al, 2001, Chimeric PSA enhancers exhibit augmented activity in prostate cancer gene therapy vectors, Gene Ther., 8:1416-1426). However, upon activation by a recombinase, a toxin gene may be driven by an even stronger promoter.

In one aspect of the invention, a viral vector such as an adenoviral vector can be used to deliver therapeutic nucleic acid constructs to patients. Viral vectors (and other nucleic acid vectors) can be tested by administering them to cells in culture, to xenografts, and/or to autochthomous tumors in TRAMP mice and/or other transgenic mouse models for prostate cancer and/or other cancers. According to the invention, virally-delivered PSE-BC chimeric promoter/enhancer together with Flp recombinase effectively targets gene expression to prostate cells. Regulated expression of DT-A in prostate cancer cells in culture and in xenografts results in the death of cells and the regression of tumor size. The detection of luciferase expression in prostate tumors in TRAMP mice following systemic adenoviral delivery of DNA demonstrates that this strategy successfully regulates gene expression in the whole organism. In one embodiment, non-invasive imaging of prostate tumors in TRAMP mice before and after treatment (e.g., microCAT scans) can be used to monitor disease treatment. Accordingly, viral vectors can be used for administration for treating any cancer. In some embodiments, multiple administrations of the therapeutic DNAs over time improve the effectiveness of the therapy. Accordingly, some delivery systems avoid potential immunological response problems associated with multiple viral treatments.

In one aspect of the invention, an expression vector is provided. In one embodiment, the expression vector includes a tissue specific regulatory element operably joined to a nucleic acid encoding Flp recombinase and a nucleic acid encoding diphtheria-toxin. The tissue specific regulatory element may include an enhancer/promoter sequence of a prostate specific antigen. In another embodiment, the nucleic acid encoding diphtheria-toxin, encodes diphtheria-toxin subunit A. An example of such a construct is provided by SEQ ID NO: 1 which includes sequence required for Flp recombinase expression in prostate cells and for DTA expression in response to threshold levels of Flp recombinase.

According to aspects of the invention, a nucleic acid or expression vector may be transferred or transfected into a cell (a linear nucleic acid such as one having the sequence shown in SEQ ID NO: 1 may be used directly or it may be incorporated into a vector as described herein). A nucleic acid such as a vector may be delivered using a viral or a non viral technique (e.g., in combination with a synthetic polymer). In another embodiment, a nucleic acid or expression vector may be administered locally to a cell, tissue or organ by any method known to those of ordinary skill in the art. Local delivery, for example, can be by injection directly to the cell, tissue or organ.

In one aspect of the invention, a method for treating cancer is provided. In one embodiment, a cell is contacted with the expression vector. In a second embodiment, the cell is a cancer cell. In a further embodiment the cancer cell is selected from the group consisting of biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; esophageal cancer; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

It should be appreciated that methods and compositions described herein include using two or more recombinases and/or toxin encoding genes, as the invention is not limited in this respect.

### BRIEF DESCRIPTION OF THE DRAWINGS

FIG. 1. DNA constructs.

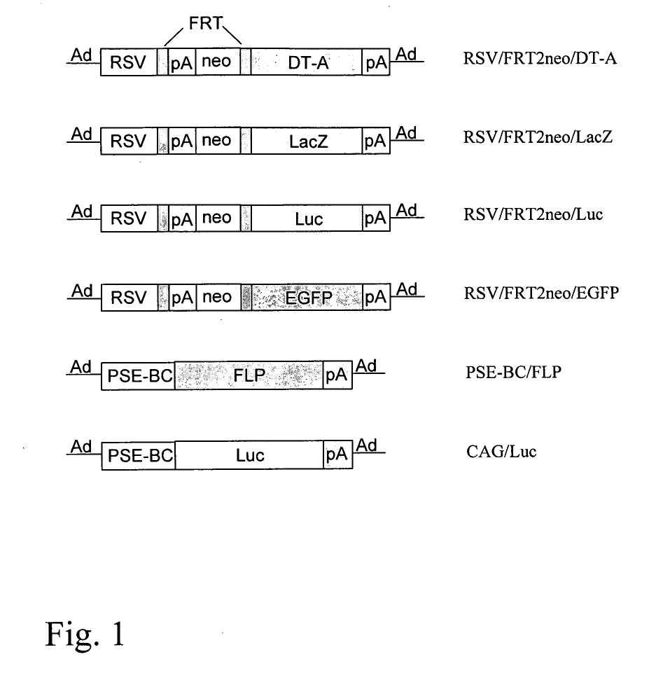

FIG. 2. PSE-BC-driven Flp recombinase-mediated regulation of EGFP in PC-3 cells and LNCaP cells. Cells were infected with Ad-RSV/FRT2neo/EGFP (moi=100) and with Ad-PSE-BC/FLP (moi=10, 20, or 50) and observed by fluorescent micrography 48 hours after viral infection.

FIG. 3. PSE-BC-driven Flp recombinase-mediated regulation of lacZ in LNCaP cells. FIGS. 3a and 3b: Cells were infected sequentially with Ad-RSV/FRT2neo/lacZ and Ad-PSE-BC/FLP. Following infection, cells were cultured in the absence (FIG. 3a) or presence (FIG. 3b) of 1 nM R1881 for 48 hours. Then, they were histochemically stained for β-gal activity. FIG. 3c: Non-infected cells cultured in the presence of 1 nM R1881.

FIG. 4. Death of PSA-expressing cells in culture resulting from PSE-BC-driven Flp recombinase-mediated activation of DT-A. Cells from each line were infected with two mixtures of viruses: Ad-PSE-BC/FLP+Ad-RSV/FRT2neo/DT-A (gray bars), and Ad-PDE-BC/FLP+Ad-RSV/FRT2neo/LacZ (black bars). Infected cells were then transfected with pCAG/luc. Luciferase activity, an indirect measure of cell death, was measured 24-hrs later. A. Luciferase activity in LNCaP cells. The multiplicity of infection (moi) of Ad-PSE-BC/FLP varied as indicated. B. WI-38 cells. Human smooth muscle cells.

FIG. 5. PSE-BC-driven Flp recombinase-mediated regulation of EGFP in xenografts following intratumoral administration of the indicated viruses. Forty-eight hours following injection of viruses, tumors were excised from the host and observed as whole mounts under fluorescent light. In FIG. 5b, the buffer in which the viruses were delivered contained 4 nM R1881. FIG. 5g: Western blot analysis of protein extracts prepared from xenografts injected with viruses. Lanes 1-8,5-days post-injection; lanes 10-13, 9-days post-injection. Lane 1, Ad-RSV/FRT2neoLacZ; lane 2, Ad-PSE-BC/FLP+RSV/FRT2neoLacZ; lane 3, Ad-RSV/FRT2/puro+Ad-RSV/FRT2neoEGFP; lane 4, Ad-PSE-BC/FLP+Ad-RSV/FRT2neoEGFP; lane 5, Ad-PSE-BC/FLP+RSV/FRT2neoEGFP+R1881; lane 6, Ad-CAG/EGFP (10⁸); lane 7, Ad-CAG/EGFP (10⁹); lane 8, Ad-CAG/EGFP (10¹⁰); lane 9, liver from CAG/EGFP transgenic mouse (positive control); lane 10, Ad-PSE-BC/FLP+Ad-RSV/FRT2neo/EGFP; lane 11, Ad-PSE-BC/FLP+Ad-RSV/FRT2neo/LacZ; lane 12, Ad-RSV/FRTpuro+Ad-RSV/FRT2neo/EGFP; lane 13, Ad-PSE-BC/FLP+Ad-RSV/FRT2neo/EGFP+R1881. The arrow indicates 27 kD EGFP (green bands). The single 45 kD red band in each lane is β-actin, controlling for amount of protein loaded. 20 μg total protein loaded per lane.

FIG. 6. Reduction in tumor volume following PSE-BC-driven Flp recombinase-mediated regulation of DT-A. Xenografts deriving from LNCaP cells were injected intratumorally two times with Ad-PSE-BC/FLP+Ad-RSV/FRT2neoDT-A (Flp/DTA) or Ad-PSE-BC/FLP+Ad-RSv/FRT2neoLacZ (Flp/LacZ). At the time of the first injection and 6-days following the second injection, the size of each tumor was determined using calipers. The mean tumor volume of each treatment group is indicated by a horizontal line.

FIG. 7. A. Synthesis and structure of polymer C32. B. Transfection efficiency of different poly(β-amino ester)polymer formulations. Various polymer formulations (C32, F28, and U94) were used to make nanoparticles for delivery of a luciferase reporter construct, pCAG/luc, to 293 cells in culture. (CAG is a very strong, ubiquitously expressed promoter/enhancer). The polymer:DNA ratio was 30:1 for all polymers tested. Cells were also transfected using Lipofectamine 2000 (Gibco-BRL) and PEI for comparison. After a 3-hr incubation with nanoparticles, the medium was changed. After 48 hours in culture, cells were harvested, and protein extracts were prepared and assayed for luciferase activity. Experiment was repeated three times. lipo=Lipofectamine 2000.

FIG. 8. Nanoparticle delivery of DNA constructs to LNCaP cells in culture. The DNA construct delivered to cells is indicated under each panel. Cells that express GFP fluoresce green. The constructs are illustrated on the right-hand side of the figure (FIG. 8e) and described in detail in the text. The way in which Flp recombinase-mediated DNA-recombination results in activation of EGFP expression is also illustrated. After transfection with C32-pRSV/FRT2.PSAFlp/EGFP, cells in FIG. 8c were cultured in medium lacking R1881, while cells in FIG. 8d were cultured in medium containing 1 nM R1881. pA=polyadenylation site. Small arrows above RSV and PSA boxes indicate the direction of transcription.

FIG. 9. Inhibition of luciferase enzyme activity by nanoparticle-delivered DT-A. LNCaP cells were treated with C32-DNA nanoparticles for 1 hour after which the medium was changed. The DNA constructs used are below each bar. After 48 hours in culture, cells were harvested, and protein extracts were prepared and assayed for luciferase activity. luc=C32-pCAG/luc, EGFP=C32-pRSV/FRT2.PSAFlp/EGFP, DT-A=C32-pRSV/FRT2.PSAFlp/DT-A. The experiment was repeated three times.

FIG. 10. EGFP expression in xenografts following injection with C32-pCAG/EGFP nanoparticles. Three whole mount tumors viewed by fluorescent microscopy.

FIG. 11. Tumor growth following intratumoral injection of C32-salmon sperm DNA or C32-pRSV/FRT2.PSAFlp/DT-A nanoparticles. Nanoparticles were injected on day 0, day 3, and day 10 (50 μg DNA/injection, 30:1 polymer:DNA ratio). Tumor volume was measured using calipers on day 0 and day 11. Fold increase in tumor volume is the ratio of these two measurements. n=5 for each group.

FIG. 12. Poly(β-amino ester)amino (6-94) and acrylate (B-LL) monomers.

### DESCRIPTION OF SEQUENCES

SEQ ID NO:1−Nucleic acid sequence of a DTA construct.

### DETAILED DESCRIPTION OF THE INVENTION

The invention provides methods and compositions for treating cancer. In particular, the invention provides nucleic acid molecules that are cytotoxic or cytostatic. In one embodiment, the invention is useful to kill cancer cells. In another embodiment, the invention is useful to slow, stop, or prevent cancer cell growth or proliferation. The invention is also useful to treat metastasis (e.g., to prevent or reduce cancer metastasis and/or kill or slow the growth of metastatic cells). In one aspect of the invention, methods result in the destruction of a non-vital tissue or organ (or portion thereof) that contains both cancerous and non-cancerous cells.

Nucleic acid molecules of the invention include a toxic gene, the expression of which can be activated by a nucleic acid recombinase. The toxic gene is not expressed in the absence of recombinase, or if the amount of recombinase is below a threshold level required for the recombination event that leads to expression of the toxic gene. According to the invention, the recombinase is expressed from a nucleic acid molecule and the expression of the recombinase is under the control of one or more cancer-associated factors.

In one embodiment, a cancer-associated factor may be a factor that is present only in cancer cells. This results in expression of the toxin only in cancer cells. In another embodiment, a cancer associated factor may be a factor that is selectively present in all cells of a certain cell type if the cancer cells (or other diseased cells) are cells of that cell type. This results in expression of the toxin in all cells of the cell type including the diseased cells (e.g., the cancer cells) and the healthy cells of the cell type. A nucleic acid of this embodiment may be delivered systemically if the cell type is non-vital (e.g., the cell type is a cell type of a non-vital organ). Alternatively, a nucleic acid of this embodiment may be delivered locally (e.g., by injection) to a diseased area of any tissue (including vital tissues and non-vital tissues).

A cancer-associated factor may be a factor that affects one or more of the replication, transcription, translation, folding, modification, or other activation of the recombinase. In one embodiment, a cancer-associated factor may be a promoter that is selectively active (e.g., results in expression) in a non-vital tissue or organ that contains a cancer (e.g., a prostate specific promoter that is active in prostate tissue that may contain a cancer).

Accordingly, in preferred embodiments, a recombinase gene is operably joined to a cancer-specific or a cell type-specific or tissue-specific promoter. In contrast, the toxin gene may be expressed from any type of promoter (including a tissue-specific, a cancer-specific, a cell type-specific, or a non-specific promoter) provided that the toxin gene is operably joined to the promoter only after a recombination event mediated by the recombinase (and the recombinase is expressed at sufficient level to mediate the recombination event only in the cells that selectively express the recombinase).

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide. In some embodiments, the expression of the recombinase is under the control of a promoter that is more active in cancer cells than non-cancer cells. This promoter can be a promoter that is only active in cancer cells, alternatively this promoter can be active in all cells but is more active in cancer cells. For example, the promoter can be a promoter from a gene that is over-expressed in cancer. Cancer over expressed promoter examples include Bcl2. In some embodiments, the promoter is a cancer-specific promoter in that it is active (or its activity is increased) only in specific types of cancers (e.g. cancer of specific cell or tissue types). However, the promoter also can be a promoter that is active (or has increased activity) in several cancer cell or tissue types. In other embodiments, a promoter and/or regulatory element can be a tissue or organ specific promoter and/or regulatory element. In one embodiment, the promoter may be selectively active in a specific cell type that includes cancerous and non-cancerous cells (e.g. prostate epithelial cells, some of which may be cancerous). Promoters may be natural promoters or synthetic or chimeric or hybrid promoters (including promoters associated with synthetic or chimeric or hybrid enhancer or other regulatory elements).

Natural promoters can be strengthened by combining them with or adding portions from strong promoters that are useful for expression in mammalian cells. Accordingly, in some nucleic acid constructs the recombinase is under the control of a promoter region that includes a cancer-associated promoter (or portion thereof) and a second promoter (or portion thereof). In other embodiments, either one or both promoters can be tissue or organ specific. Examples of useful systems for mRNA expression in mammalian cells are those such as pRc/CMV and pcDNA3.1 (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences.

In some embodiments, nucleic acid constructs of the invention may be contained on expression vectors. These vectors may be able to replicate in mammalian cells. However, in some embodiments, a vector can be amplified in cells of one organism (e.g., bacterial, insect, yeast, or mammalian cells) but does not replicate in cells of the organism (e.g., a mammal such as a human) that is being targeted. A suitable vector for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Other examples of an expression vector include an adenovirus, described by Stratford-Perricaudet, which is defective for E1 and E3 proteins (J. Clin. Invest. 90:626-630, 1992). The use of the adenovirus as an Adeno.P1A recombinant is disclosed by Warnier et al., in intradermal injection in mice for immunization against P1A (Int. J. Cancer, 67:303-310, 1996). Recombinant vectors including viruses selected from the group consisting of adenoviruses, adeno-associated viruses, poxviruses including vaccinia viruses and attenuated poxviruses such as ALVAC, NYVAC, Semliki Forest virus, Venezuelan equine encephalitis virus, retroviruses, Sindbis virus, Ty virus-like particle, other alphaviruses, lentiviruses, VSV, plasmids (e.g. “naked” DNA), bacteria (e.g. the bacterium Bacille Calmette Guerin, attenuated Salmonella), and the like can be used for such delivery, for example, for prophylactic use.

According to aspects of the invention, one or more nucleic acid constructs (including, for example, one or more of the vectors described above) may be delivered using viral methods (e.g., retroviral, adenoviral, lentiviral), or using non-viral methods (e.g., polymeric nanoparticles, liposomes, naked DNA).

The recombinase is preferably an enzyme that catalyzes a specific type of nucleic acid recombination event, preferably involving one or more specific nucleic acid recognition sequences. Recombinase examples include FLP recombinase and other recombinase enzymes (e.g., Cre recombinase and Hin recombinase which act on their own specific sites; Cre recombinase is a Type I topoisomerase from bacteriophage P1 that acts on lox (loxp) site; Hin recombinase acts on the flagellin genes hixL and hixR from Salmonella). The recombinase can be a monomer, dimer, heterodimer, multimer, or heteromultimer. In the embodiments where the recombinase requires two or more different subunits, the expression of at least one of the subunits is under that control of a cancer-associated factor such as one of the factors described herein, including tissue or organ specific. In some embodiments, the recombinase can be one or more proteins, nucleic acids, or a combination thereof that catalyze a specific recombination event.

The toxic gene can be a cytostatic or cytotoxic gene. Toxic gene examples include bacterial toxins for example diptheria toxin, and other toxins. Toxins can also be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60. The toxic gene can encode a toxic nucleic acid (e.g. an RNA such as an antisense or interfering RNA), or a toxic protein such as a toxic enzyme, or a combination thereof. The toxic nucleic acid or protein can be a monomer, dimer, heterodimer, multimer, or heteromultimer. In the embodiments where the toxic nucleic acid or protein requires two or more different subunits, the expression of at least one of the subunits is under that control of the recombinase. The control of the toxic gene expression by the recombinase can involve a recombinase catalyzed rearrangement of the structural elements (or one or more portions thereof) or of one or more regulatory elements (e.g. promoter, enhancer, splicing sequence, terminator, etc.) controlling expression of the structural elements of the toxic gene.

The recombinase and the toxic gene can be encoded on the same nucleic acid or on different nucleic acids. If either of the recombinase or toxic genes requires two or more subunits, these can be encoded on the same or additional nucleic acids. The nucleic acid is preferably a DNA molecule. The DNA molecule can be a circular plasmid. The DNA molecule can be a linear molecule. The nucleic acid molecule can be self-replicating. The nucleic acid molecule can be based on a viral genome and include the sequences required for viral replication (e.g. DNA or RNA replication) or can include sequences for subgenomic RNA amplification. The nucleic acid molecule can be based on a plasmid with an origin of replication. The nucleic acid molecule can be a molecule that is stable or replicates intracellularly. The nucleic acid molecule can also be a molecule that integrates into the genome (including the mitochondrial genome) of a cell. A nucleic acid may be RNA or DNA and may be of any size or sequence, double-stranded or single-stranded. A nucleic acid may be purified from contaminating components using methods known to those of ordinary skill in the art. However, a nucleic acid also can be a synthetic stable nucleic acid.

The invention provides methods for treating cancer cells. Accordingly, the invention provides methods for treating cancer patients. Methods are also provided for treating cancer in non-vital organs or tissues. Methods of treating may also include ablation or destruction of a whole tissue or organ if the tissue or organ is non-vital. Methods of treating further include local delivery to an area of a tissue or organ. In some embodiments the area of delivery may include diseased (e.g., cancerous) and healthy cells. Cancers include but are not limited to biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; esophageal cancer; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

Methods of the invention also are useful for treating other disorders, for example in patients with benign prostatic hyperplasia (BPH) it may be desirable to reduce the size of the prostate which could be achieved with local delivery of the construct of the invention. The methods of the invention are also useful for treating benign tumor growths.

Aspects of the invention include administering one or more molecules of the invention to a subject that has or is at risk of developing cancer. A subject having cancer is a subject that is diagnosed with one or more cancers using known diagnostic methods. A subject at risk of developing cancer is a subject that has one or more known risk factors associated with cancer. Known risk factors include a genetic predispositions, cancer-associated mutations detected in a biological sample, environmental risk factors, exposure to carcinogenic agents, age, inflammatory diseases, and other factors associated with cancer. Examples include immunodeficiency or viral associated cancers. Subjects include mammalian subjects. Preferred subjects are human subjects. Other mammalian subjects include dog, cat, mouse, horse, cow, goat, sheep, pig and other mammals.

Accordingly, aspects of the invention also include methods to prevent the onset, progression, or increase the regression of disorders associated with uncontrolled cell-growth. Onset of a cancer is the initiation of the physiological changes or characteristics associated with the cancer condition in a subject. Such changes may be evidenced by physiological symptoms, or may be clinically asymptomatic. For example, the onset of a disorder associated with abnormal cellular proliferation may be followed by a period during which there may be physiological characteristics in the subject, even though clinical symptoms may not be evident at that time. The progression of a condition follows onset and is the advancement of the physiological characteristics of the condition, which may or may not be marked by an increase in clinical symptoms. In contrast, the regression of a condition is a decrease in physiological characteristics of the condition, perhaps with a parallel reduction in symptoms, and may result from a treatment or may be a natural reversal in the condition.

Aspects of the invention also include administering one or more nucleic acids of the invention to post-operative or post-treatment (e.g. chemotherapy, radiotherapy, or hormonal therapy) cancer patients to prevent the recurrence or spreading of cancer (e.g., metastatic cancer).

Nucleic acid molecules of the invention also can be administered to a patient as part of a cancer therapy (e.g. along with surgery, chemotherapy, radiotherapy, hormonal therapy, or combinations thereof). Accordingly, the invention includes pharmaceutical compositions having both a nucleic acid of the invention and one or more additional cancer therapeutic molecules.

Accordingly, aspects of the invention include methods of administering a therapeutically effective amount of a toxic nucleic acid to a subject, wherein the therapeutically effective amount is an amount that is sufficient to prevent, reduce, cure, or alleviate the symptoms of a cancer.

Nucleic acid molecules of the invention can be maintained, replicated, amplified, and/or delivered in a vector (e.g., a viral vector) or with one or more other carrier molecules. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate autonomously or after integration into the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In some embodiments, plasmids can be replicated in bacterial systems. Replication of the desired sequence may occur many times as the plasmid increases in copy number within the host bacterium or just a single time per host before the host reproduces by mitosis. Phage systems can also be used to obtain multiple copies of a nucleic acid of the invention. Replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

A nucleic acid may be modified resulting in increased stability. Modifications include but are not limited to methylation, phosphorylation, end-capping, and modifications to the backbone. A nucleic acid includes but is not limited to a circular plasmid, a linearized plasmid, a cosmid, a viral genome, a modified viral genome or an artificial chromosome. A nucleic acid may be amplified in a suitable host organism. Alternatively, or in addition, a nucleic acid may be amplified using an in vitro amplification techniques (e.g., PCR, LCR, etc.).

Various techniques may be employed for introducing a vector of the invention containing a nucleic acid molecule of interest or for introducing the nucleic acid molecule without the vector into a cell. Such techniques include calcium phosphate precipitate transfection, DEAE transfection, transfection or infection with viruses (e.g. virus-based nucleic acids can be package in virus particles for delivery), liposome-mediated transfection (lipofection), ballistic transformation, (micro-)injection, transfection, electroporation and the like.

For certain uses, it is preferred to target the vector containing a nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the vector containing a nucleic acid molecule delivery vehicle. Especially preferred are monoclonal antibodies. Liposomes are commercially available from Life Technologies, Inc., for example, as LIPOFECTIN™ and LIPOFECTACE™, which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, 3:235-241 (1985). Where liposomes are employed to deliver the vector of the invention containing a nucleic acid molecule, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

Delivery preparations of the invention can be administered by any known method including by injection, ingestion, inhalation, and gene gun administration. The preparations can be administered systemically or locally to the area of the cancer (including injection into a tumor). The preparations can be administered via oral, mucosal, subdermal, intramuscular, intravenous, intraperitoneal and other routes. Other useful delivery methods for example, endosomolytic agents and biodegradable polymer nanospheres, are disclosed in U.S. Pat. Nos. 6,692,911, 6,254,890 and U.S. patent application number 20010036460, the disclosures of which are incorporated in their entirety herein by reference.

The preparation can be administered (e.g. injected) to any portion or area of a tissue or an organ. The preparation can be administered to a localized area of a tissue or an organ. In aspects of the invention, the preparations can be administered directly to an epithelial cell layer of a tissue or an organ. The epithelial cell layer may or may not include tumor cells. The preparation can be administered directly to a tumor cell or to a non-tumor cell. Delivery of the preparation can be in the presence or the absence of a polymer as described herein. For example, a nucleic acid of the invention can be injected into epithelial cell layers of the prostate. Injection (particularly if the injected volume is small) targeting a nucleic acid or other preparation to a tissue region or organ of interest (or to a portion thereof).

In one aspect, the invention may involve delivering a toxic construct of the invention by injection into a vital tissue or organ. A tissue-specific and/or cancer-specific promoter may be used for the recombinase in order to target a portion or area of the tissue or organ which is diseased (e.g., contains cancerous cells). However, the recombinase also may be under control of a non-tissue-specific and non-cancer-specific promoter if the construct is delivered locally in a small amount to target the cancerous cells without damaging a significant number of healthy cells.

Compositions of the invention can be administered as a single dose or in two or more doses. The doses can be administered at regular intervals, including hourly, daily, weekly, monthly, yearly, and at other regular intervals. Alternatively, the doses can be administered at intervals determined by other factors such as doctor visits or patient symptoms or diagnostic tests. A therapeutically effective amount can be an amount that is effective in a single dose or as part of a multi-dose regimen.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents, oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The preparations may be sterilized using any appropriate method including filtration, heat, chemical treatment etc.

The preparations of the invention are administered in effective amounts. An effective amount is that amount of a pharmaceutical preparation that alone, or together with further doses, stimulates the desired response. In the case of treating cancer, the desired response is inhibiting the progression of the cancer. This may involve only slowing the progression of the disease temporarily, although more preferably, it involves halting the progression of the disease permanently. A desired response can be monitored by routine methods know to those of ordinary skill in the art.

Systemic Therapies

Systemic delivery of genes that encode cellular toxins is an alternative that holds great promise for improving the ability to treat metastatic cancer (e.g., metastatic prostate cancer) effectively. In some embodiments, targeted expression of diphtheria toxin (DT), resulting in the death of prostate cancer cells, provides an attractive therapeutic option because the mechanism of action of this toxin is known (Collier, R. J., 1975, Diptheria toxin: mode of action and structure, Bacteriol. Rev., 39:54-85), and the DT gene has been cloned, sequenced, and adapted for expression in mammalian cells. Naturally occurring diphtheria toxin is made by Corynebacterium diphtheriae as a secreted precursor polypeptide that is then enzymatically cleaved into two fragments, the A and B chains. The B chain binds to the surface of most eukaryotic cells and then delivers the A chain (DT-A) into the cytoplasm. Once inside the cell, DT-A inhibits protein synthesis. It is extremely toxic; a single molecule is sufficient to kill a cell (Yamaizumi, M. et al, 1978, One molecule of diphtheria toxin fragment A introduced into a cell can kill the cell, Cell, 15:245-250). A DT gene, engineered for use in mammalian cells, DT-A, encodes the DT-A subunit, but not the DT-B subunit (Maxwell, I. H. et al., 1986, Regulated expression of a diphtheria toxin A-chain gene transfected into human cells: possible strategy for inducing cancer cell suicide, Cancer Res., 46:4660-4664). The DT-A subunit is retained within the cytoplasm of the cell. In the absence of the B subunit, even DT-A released from dead cells is not able to enter other neighboring cells, thereby ensuring that the toxin only kills targeted cells.

Some embodiments of the invention make use of the fact that prostate specific antigen (PSA) is expressed, as its name suggests, specifically in prostate cells. PSA is expressed by luminal cells in the epithelium of the normal prostate and usually by prostate tumor cells, irrespective of their dependency on androgen. Preferably, a chimeric modified enhancer/promoter sequence of the human prostate specific antigen (PSA) gene, PSE-BC (Wu, L et al., 2001, Chimeric PSA enhancers exhibit augmented activity in prostate cancer gene therapy vectors, Gene Ther., 8:1416-1426), is used to regulate the expression of Flp recombinase that, in turn, activates DT-A expression. In some embodiments, adenoviral delivery of Flp recombinase, reporter gene, and DT-A DNA constructs to cells in culture and to xenografts deriving from human prostate cancer cells, was used to assess the ability of the dual-regulatory control strategy to target gene expression to prostate cells. Significantly, a 50% reduction in tumor size was observed following PSE-BC-directed activation of DT-A expression. This shows that systemic administration of genes incorporating the PSE-BC enhancer/promoter together with Flp recombinase-mediated DNA recombination allow for targeted expression of DT-A to prostate cells. Accordingly, this approach can be useful as an effective therapy for metastatic prostate cancer. Other tissue-specific and/or cancer-specific expression elements may be used to target expression of a toxic element to other tissues that contain cancer cells, particularly non-vital tissues or organs as described herein.

In preferred clinical applications, nucleic acids of the invention are delivered using safe and efficient delivery systems for systemic gene transfer. In some embodiments, the ability to target expression of a toxin to PSA-expressing cells allows for systemic delivery of therapeutic DNA to patients with metastatic prostate cancer. All cancerous lesions derived from a specific cell type should be effectively treated without any significant adverse effects on non-cancerous, healthy tissue, of other cell types.

In preferred embodiments of the present invention, all of the genetic elements required for the regulatory system are housed in one nucleic acid molecule (e.g. a single DNA molecule) rather than two or more molecules. For example, PSA promoter-driven Flp recombinase can be embedded within the same DNA construct as the DT-A sequence, serving as the intervening DNA that silences the expression of DT-A in non-PSA-expressing cells. Embedded nucleic acid constructs of the invention can be very large and may be difficult to deliver using standard viral vectors for DNA delivery. Accordingly, non-viral vectors, such as polymeric nanoparticles, may be used for delivering large nucleic acid molecules such as certain DNA molecules of the invention.

Non-Viral Delivery

Many delivery systems currently used are based on viral delivery systems, which while generally efficient at DNA delivery, can suffer from potentially serious toxicity and production issues (Somia, N. et al., 2000, Gene therapy trials and tribulations, Nat. Rev. Genet., 1:91-99). Synthetic DNA delivery systems, in contrast, offer a significant number of advantages, including stability, ease and cost of production, low immunogenicity and toxicity, and reduced vector size limitation (Ledley, F. D. et al., 1995, Nonviral gene therapy: the promise of genes as pharmaceutical products, Human Gene Ther., 6:1129-1144). Despite these advantages, however, many existing non-viral delivery systems are far less efficient than viral vectors (Luo, D., et al., 2000. Synthetic DNA delivery systems, Nat. Biotechnol., 18:33-37).

Many of the leading non-viral delivery compounds are cationic polymers, which can spontaneously bind and condense DNA into nanoparticles. A wide variety of cationic polymers that transfect cells in vitro have been characterized; some are natural polymers such as protein (Fominaya, J., et al., 1996, Target cell-specific DNA transfer mediated by a chimeric multidomain protein—novel non-viral gene delivery system, J. Biol. Chem., 271:10560-10568) and peptide systems (Schwartz, J. J., et al., 2000, Peptide-mediated cellular delivery, Curr. Opin. Mol. Ther., 2:162-167), others are synthetic polymers such as poly(ethylene imine) (PEI) (Boussif, O., et al., 1995, A versatile vector for gene and oligonucleotide transfer into cells in culture and in vivo—polyethyleninine, Proc. Natl. Acad. Sci. USA, 92:7297-7301) and dendrimers (Kabanov, A. V., et al., 1998, Self-assembling complexes for gene delivery: from laboratory to clinical trial, Wiley, Chichester, N.Y.). Recent advances in polymeric gene delivery have in part focused on the ability of polymers to biodegrade, thereby decreasing toxicity. Typically, these polymers contain both chargeable amino groups, to allow for ionic interaction with the negatively charged DNA phosphate, and a degradable region, such as a hydrolyzable ester linkage. Several examples of these include poly(alpha-(4-aminobutyl)-L-glycolic acid) (Lim, Y. B., et al., 2000, Development of a safe gene delivery system using biodegradable polymer, poly[alpha-(4-aminobutyl)-L-glycolic acid], J. Am. Chem. Soc., 122:6524-6525), network poly(amino ester) (Lim, Y. B. et al., 2002, Biodegradable, endosome disruptive, and cationic network-type polymer as a highly efficient and nontoxic gene delivery carrier, Bioconjugate Chem, 13:952-957), and poly(β-amino ester)s (Lynn, D. M., et al., 2000, Degradable poly(beta-amino esters): Synthesis, characterization, and self-assembly with plasmid DNA, J. Am. Chem. Soc., 122:10761-10768; Lynn, D. M., et al., 2001, Accelerated discovery of synthetic transfection vectors: Parallel synthesis and screening of a degradable polymer library, J. Am. Chem. Soc., 123:8155-8156). In preferred embodiments, nucleic acid constructs of the invention can be effectively delivered by poly(β-amino ester)s to human cancer cells such as prostate cancer cells.

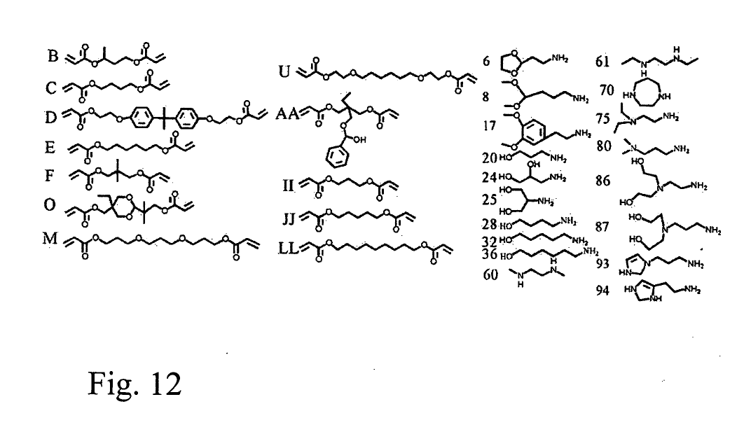

As used herein, the term “cationic polymer” refers to any polymer or a portion thereof with a net positive charge. The cationic polymers include poly(β-amino ester)s, such as those described herein, including A5, A8, A11, AA24, AA20, JJ20, AA28, B6, B9, B11, B14, C4, C12, C28, D6, D24, D60, E7, E14, E20, F20, G5, C32 (e.g., C32-2), O20, U28 (e.g., U28-3), JJ20, SJJ28 (e.g., JJ28-3), D94 (e.g., D94-5), E28 (e.g., E28-3), U32 (e.g., U32-2), JJ32 (e.g., JJ32-3), F28 (e.g., F28-6) and F32 (e.g., F32-2). These polymers include an acrylate (e.g., diacrylate) indicated by the letter (see FIG. 12) and an amine indicated by the number (see FIG. 12). Typically, these polymers have one or more tertiary amines in the backbone of the polymer. Poly(β-amino ester)polymers may also be copolymers in which one of the components is a poly(β-amino ester). These polymers can be prepared, for example, by condensing bis(secondary amines) or primary amines with bis(acrylate esters). Poly(β-amino ester)s and methods of their production are also described in U.S. Patent Application publications 20020131951 published Sep. 19, 2002, and 20040071654 published Apr. 15, 2004, both incorporated herein by reference. The structures for a library of 94 poly(β-amino ester)s as well a methodology for their synthesis can be found in Anderson et al., “Semi-Automated Synthesis and Screening of a Large Library of Degradable Cationic Polymers for Gene Delivery”, Angew. Chem. Int. Ed. 2003, 42, 3153-3158. A library of 140 poly(β-amino ester)s is described in Lynn et al., “Accelerated Discovery of Synthetic Transfection Vectors: Parallel Synthesis and Screening of a Degradable Polymer Library”, J. Am. Chem. Soc. 2001, 123, 8155-8156.

In some embodiments, the polymers described above are prepared using a ratio of between 1/1 and 5/1 (e.g., about 2/1) of amine to acrylate in order to produce a polymer with a net positive charge. For example, C32 may be prepared with a ratio of amine to acrylate of about 1.2/1. However, other ratios may be used. These polymers may be complexed to nucleic acid constructs of the invention by incubating a mixture of one or more polymers with one or more nucleic acid constructs at a pH below 7.0 (e.g., in an acetate buffer at about pH 5.0) followed by neutralization using a buffer such as saline or PBS or other physiologically compatible buffer. In one embodiment, the ratio of polymer to nucleic acid may be between 10/1 to 100/1 by weight (e.g., about 30/1, or about 40/1 of polymer to DNA). However, other ratios may be used. The resulting product may be administered to a subject as described herein.

Cationic polymers can also include natural cationic polymers, such as proteins and peptides or synthetic cationic polymers, such as poly(ethylene imine) (PEI). In some embodiments, the cationic polymer is degradable. Degradable cationic polymers can contain both chargeable amino groups, to allow for ionic interaction with the negatively charged polysaccharides, and a degradable region, such as a hydrolyzable ester linkage. Examples of these include poly(alpha-(4-aminobutyl)-L-glycolic acid), network poly(amino ester), polyethylene imine, polylysine, polyarginine and poly(-amino ester)s as provided above. In other embodiments the cationic polymer is rapidly degradable. “Rapidly degradable” as used herein refers to the relatively short amount of time required to break down the cationic polymer into its constituent parts. The speed of degradability can be assessed by comparison, for instance, to polylysine. In some embodiments, a rapidly degradable polymer is one that is degraded faster than polylysine under the same conditions. The degradation may be by enzymatic or hydrolytic degradation. In yet other embodiments, the cationic polymer is a cationic polymer as defined above but is not a protamine, a histone, a polyamino acid, or a polyamido amine. In still other embodiments the cationic polymers as provided herein are not polyornithine or polylysine. Preferably, the cationic polymers employed in the compositions provided, particularly, those used for the intracellular delivery of polysaccharide in a subject, are cationic polymers with low toxicity. A “cationic polymer with low toxicity” is one that is less toxic than polylysine when compared in the same amount under the same conditions. In some instances a cationic polymer with toxicity greater than or equal to polylysine may be desired.

Aspects of the invention include nucleic acid constructs with different promoter sequences for regulating the expression of a recombinase (e.g., Flp recombinase) in order to target expression of a toxin to different types of cells (including cancer cells), tissues or organs. The invention is not limited to methods and compositions involving toxins, but can also be used to restrict the expression of other nucleic acids or genes encoding proteins (e.g., enzymes, structural proteins) to particular tissues. The following examples illustrate embodiments of the invention. However, the scope of the invention is not limited by these examples.

### EXAMPLES

Materials and Methods

Shuttle plasmids and virus production. To make the shuttle plasmid pPSE-BC/FLP, we first constructed pMECA-PSE-BC/FLP. A 2-kb fragment, released by digestion of pOG-FLPe6 (gift of A. Francis Stewart, EMBL, Heidelberg) with XbaI and SalI, was ligated to XbaI+XhoI-digested pMECA (Thomson, J. M. et al., 1998, A cloning plasmid with 44 unique restriction sites that allows selection of recombinants based on colony size, BioTechniques, 24:922-928) to produce pMECA/FLP(1). This plasmid was then digested with XbaI and AgeI to release a 2-kb fragment that was ligated to NheI+NgoMI digested pMECA to create pMECA/FLP (2). A 2.5-kb fragment, released from the plasmid pPSE-BC by digestion with XbaI and SalI, was ligated to XbaI+SalI-digested pMECA/FLP(2) to create pMECA-PSE-BC/FLP. A 4.5-kb fragment released from pMECA-PSE-BC/FLP by digestion with XbaI and AvrII, was ligated to XbaI-digested pDC312 (Microbix Biosystems, Inc., Toronto, Canada) to create pPSE-BC/FLP.

The shuttle plasmid pRSV/FRT2neo/lacZ was constructed as described (Peng, W. et al., 2002, Regulated expression of diphtheria toxin in prostate cancer cells, Mol. Ther., 6:537-545).

The shuttle plasmid pRSV/FRT2neo/EGFP was constructed as follows. A 1.3-kb fragment containing FRT2neo (released by digestion with AgeI from the plasmid pFRT2neolacZ, a gift of Susan Dymecki) was inserted into the AgeI site of pRSV/EGFP to create pRSV/FRT2neo/EGFP. To create pRSV/EGFP, a 1-kb fragment, released by digestion of pEGFP-1 (Clontech) with BamHI and AflII, was ligated to BamHI+AflII-digested pIND (Invitrogen) to produce pIND/EGFP. A 1-kb fragment, released from pIND/EGFP by digestion with SpeI and NheI, was then ligated to NheI-digested pDC312/RSV (Peng, W. et al., 2002, Regulated expression of diphtheria toxin in prostate cancer cells, Mol. Ther., 6:537-545) to generate pRSV/EGFP).

The plasmid RSV/FRT2PSA.FLP/EGFP was constructed as follows. A 2-kb fragment, released by digestion of pOG-FLPe6 (gift of A. Francis Stewart, EMBL, Heidelberg) with XbaI and SalI, was ligated to XbaI+XhoI-digested pMECA (Tomson, J. M., et al., 1998, pMECA: A cloning plasmid with 44 unique restriction sites that allows selection of recombinants based on colony size, BioTechniques, 24:922-928) to produce pMECA/FLP(1)(dam-). This plasmid was then digested with XbaI and AgeI to release a 2-kb fragment that was ligated to NheI+NgoMI digested pMECA to create pMECA/FLP (2)(dam-). A 2.5-kb fragment, released from the plasmid pPSE-BC (gift of Lily Wu, UCLA) by digestion with XbaI and SalI), was ligated to XbaI+SalI-digested pMECA/FLPe(2)(dam-) to create pMECA/PSA.FLP(dam-). A 4.5-kb fragment, released from pMECA/PSA.FLP(dam-) by digestion with XbaI and AvrII, was ligated to NheI-digested pFRT2 (gift of Susan Dymecki, Harvard) to generate pFRT2/PSA.FLP. Finally, this plasmid was digested with AgeI and XmnI to release a 4.5-kb fragment that was ligated to AgeI-digested pRSV/EGFP to produce RSV/FRT2PSA.FLP/EGFP.

The shuttle plasmid pRSV/FRT2neo/DT-A was constructed as follows. The plasmid p22EDT1 (gift of A. Francis Stewart, EMBL, Heidelberg) was digested with BglII and NotI, releasing a 1.3-kb fragment that was ligated to a 5.0-kb fragment released from the plasmid pIND by BamHI+NotI digestion to create pIND/DT-A. pIND/DT-A was digested with KpnI and XbaI, releasing a 1.3-kb fragment which was ligated to a KpnI+XbaI digest of pMECA [Thomson, 1998] to create pMECA/DT-A. This plasmid was digested with AgeI and XbaI, releasing a 1.3-kb fragment which was ligated to a 3.8-kb fragment deriving from an AgeI+NheI digest of pRSV/EGFP. The resulting plasmid, pRSV/DT-A, was digested with AgeI, and then ligated to a 1.3-kb fragment released by digestion with AgeI from the plasmid pFRT2neolacZ. Insert orientation was checked by digestion with XhoI.

The plasmid pRSV/FRT2PSA.FLP/DT-A was constructed as follows. The plasmid p22EDT1 (gift of A. Francis Stewart, EMBL, Heidelberg) was digested with BglII and NotI, releasing a 1.3-kb fragment that was ligated to a 5.0-kb fragment released from the plasmid pIND by BamHI+NotI digestion to create pIND/DT-A. pIND/DT-A was digested with KpnI and XbaI, releasing a 1.3-kb fragment which was ligated to a KpnI+XbaI digest of pMECA to create pMECA/DT-A. This plasmid was digested with AgeI and XbaI, releasing a 1.3-kb fragment which was ligated to a 3.8-kb fragment deriving from an AgeI+NheI digest of pRSV/EGFP. The resulting plasmid, pRSV/DT-A, was digested with AgeI, and then ligated to a 4.5-kb fragment released from pFRT2/PSA.FLP by digestion with AgeI and XmnI to create pRSV/FRT2PSA.FLP/DT-A.

The shuttle plasmid pRSV/FRT2neo/luc was constructed as follows. A 2.0-kb fragment, released from the pBCVP2G5-lucNSN (gift of Michael Carey, UCLA) by digestion with BglII and SalI, was ligated to (BglII+SalI)-digested pMECA to create pMECA/luc. This plasmid was digested with AgeI and SalI to release a 2.0-kb fragment that was ligated to (AgeI+XhoI)-digested pDC312-RSV/EGFP to create pDC312-RSV/luc. A 1.3-kb fragment, released from the plasmid pFRT2neolacZ by digestion with AgeI, was iagated into AgeI-digested pRSV/luc (see above) to generate pRSV/FRT2neo/luc.

The plasmid pPSA/EGFP was constructed as follows. A 2.5-kb fragment was released from pPSE-BC by digestion with XbaI and SalI. This fragment was ligated to a XbaI+SalI digest of pMECA to create pMECA/PSA. pMECA/PSA was digested with SalI, BglII, and XmnI to release a 2.5-kb fragment which was then ligated to SalI+BglII-digested pEGFP-1 to create pPSA/EGFP.

The plasmid pCAG/luc was constructed as follows. A 1.2-kb fragment, released by digestion of phRL-null (Promega) with BglII and XbaI, was ligated to BglII+XbaI digested pcDNA3.1 (Invitrogen) to create pcDNA3.1/luc. The plasmid pCX-EGFP (gift of J. Miyazaki, Kyoto U.) was digested with SalI and EcoRI to release a 1.7-kb fragment. This fragment was ligated to a XhoI+EcoRI digest of pcDNA3.1/luc to produce pCAG/luc.

Ad-5 replication-defective viral vectors were prepared using the AdMax System (Microbix Biosystems, Inc., Toronto, Canada) as described (Peng, W. et al., 2002, Regulated expression of diphtheria toxin in prostate cancer cells, Mol. Ther., 6:537-545).

Salmon testes DNA (Sigma, cat. # D-1626) served as a negative control in xenograft experiments.

Cells. PC-3 cells, purchased from the American Type Culture Collection (ATCC #1435), were maintained at 37° C. in 5% CO₂, balance air, in Dulbecco's modified Eagle's medium (DMEM) supplemented with 10% fetal bovine serum. 293 cells (Microbix, Inc.) were cultured in Modified Eagle's Medium (MEM) supplemented with 10% fetal bovine serum (FBS). LNCaP cells (Urocor, Inc., Oklahoma City, Okla.) were maintained in RPMI 1640 medium supplemented with 10% FBS. Cells were maintained at 37° C. in 5% CO₂, balance air.

WI-38 cells were maintained in modified Eagle's medium (MEM), supplemented with 10% fetal bovine serum. Human smooth muscle cells from coronary artery (Cambrex, East Rutherford, N.J.) were maintained in SmGM®-2 medium (Cambrex) supplemented with 10% fetal bovine serum.

For experiments, cells were seeded at a density of 2×10⁴ cells per cm² in 24-well plates. After 48 hours, cells were sequentially infected with two viruses (first with one virus, and 16 hours later with the second virus). In some experiments, the culture medium contained 1 nM methyltrienolone (R1881) (Perkin Elmer Life Sciences, Wellesley, Mass.).

Reporter Gene Assays (X-Gal staining, luciferase, EGFP). To detect β-gal activity, cells were fixed with 4% paraformaldehyde, and then washed three times with phosphate-buffered saline (PBS) and histochemically stained [Allen, 1988]. We used the Luciferase Assay System (Promega, Madison, Wis.) and a Monolight 2010 luminometer (Analytical Luminescence Laboratory, San Diego, Calif.) to measure luciferase activity in cell extracts that were prepared according to the manufacturer's instructions.

EGFP fluorescence was detected in cells and tumors using a Zeiss Axiovert 200M inverted fluorescent microscope outfitted with a Axiocam MRm camera and Axiovision 4.0 software.

Xenografts. We inoculated 2×10⁶ LNCaP cells in PBS and 20% Matrigel (Beckton Dickinson, Bedford, Mass.) subcutaneously into each flank of 6- to 8-week old nu/nu male mice (Harlan Sprague Dawley, Indianapolis, Ind.). When tumors were approximately 250 mm³ in size, we delivered virus to the tumors by intratumoral injection (10¹⁰ viral particles of each virus). In some instances, as indicted in the text, the viral injection buffer contained 4 nM R1881 (Perkin Elmer Life Sciences, Wellesley, Mass.). In experiments with EGFP virus, tumors were excised and observed by fluorescent micrography at the indicated times. In experiments to assess tumor growth, we used calipers to measure the size of tumors before and after viral treatment. All measurements were made on intact mice.

Western Blot Analysis. Tissues and tumors were homogenized in RIPA buffer and centrifuged at 12,000 rpm for 15 min at 4° C. Protein concentration of extracts was determined using the BCA Protein Assay Kit (Pierce, Rockford, Ill.). Soluble proteins were separated by electrophoresis on 10% SDS-PAGE gels and analyzed by Western blotting. Filters were incubated with a mixture of primary antibodies, a polyclonal to EGFP (1:2000) (Clontech, Palo Alto, Calif.) and a monoclonal to β-actin (1:20,000) (Sigma, clone AC-15), washed several times with PBST (phosphate buffered saline, 0.1% Tween), and then incubated in the dark with a mixture of secondary antibodies, Alexa 680-labeled goat anti-mouse (1:25,000) and IRD800-labeled goat anti-rabbit (1:10,000). After several washes in PBST and PBS, filters were scanned using an Odyssey Infrared Imager (Li-Cor, Inc., Lincoln, Nebr.). Fluorescent signals were visualized using Odyssey VI.1 software.

Polymeric nanoparticles. C32 polymer was synthesized as previously described (Anderson, 2003 Angewandte). Cells to be transfected with nanoparticles are seeded in 96-well plates (5×10³ cells/well) and cultured for 2 days. To transfect cells with nanoparticles, the polymer is dissolved in dimethyl sulfoxide (100 mg/ml). 1.5 μg DNA is suspended in 25 μl 25 mM sodium acetate buffer, pH 5.0, and mixed with C32 polymer, also diluted in 25 μl 25 mM sodium acetate buffer, pH 5.0. The amount of polymer can vary to achieve the desired polymer:DNA weight ratio. After the polymer/DNA mix incubates for 5-10 minutes, 30 μl of the mix is diluted to 200 μl in Optimem medium (Invitrogen Corp.), and then the medium on cells is removed and replaced with 150 μl of this dilution (˜600 ng of DNA). Cells are incubated with the nanoparticles for 1 or 3 hours and then the medium is replaced with fresh growth medium (containing 10% FBS).

To prepare nanoparticles for intratumoral injection, basically the same protocol is followed excepting that instead of suspending 1.5 μg of DNA in 25 μl 25 mM sodium acetate buffer, pH 5.0, the amount of DNA is 50 μg. Also, 50 μl phosphate buffered saline (PBS) is added to the 50 μl polymer/DNA mix just prior to injection of 100 μl into the tumor. Nanoparticles are injected using a 26-gauge needle.

Xenografts. 2×10⁶ LNCaP cells in 50% Matrigel/50% PBS were injected subcutaneously into each flank of 6-week old nu/nu male mice (Harlan Sprague Dawley). Calipers were used to measure the length, width, and height of tumors. Mice were used in accordance with protocols approved by the Lankenau Institutional Animal Care and Use Committee.

Luciferase Assay. Cell extracts were prepared and assayed for luciferase activity using the Renilla Luciferase Assay System according to manufacturer's instructions (Promega).

### Example 1

### Correlation of PSE-BC Promoter-Driven Flp Recombinase-Mediated Gene Expression with PSA Expression and Androgen Level

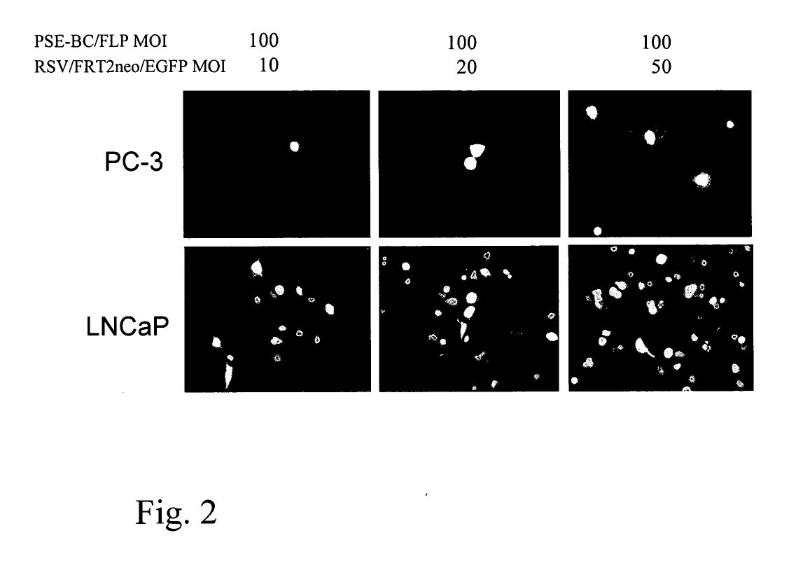

To test whether the amount of Flp recombinase-mediated gene expression driven by the PSE-BC promoter corresponds to the amount of PSA produced by cells, we infected cells from two human prostate cancer cell lines, PC-3 and LNCaP, known to differ in their expression of PSA. LNCaP cells express PSA at a high level compared to PC-3 cells. In this experiment, we infected cells with Ad-RSV/FRT2neo/EGFP (multiplicity of infection (moi)=100) and varying amounts of Ad-PSE-BC/FLP (moi=10, 20, and 50). Forty-eight hours post-infection, many more LNCaP cells expressed EGFP than did PC-3 cells (FIG. 2). Higher infectivity of both cell-types with Ad-PSE-BC/FLP resulted in more Flp recombinase-mediated recombination and more EGFP-expressing cells.

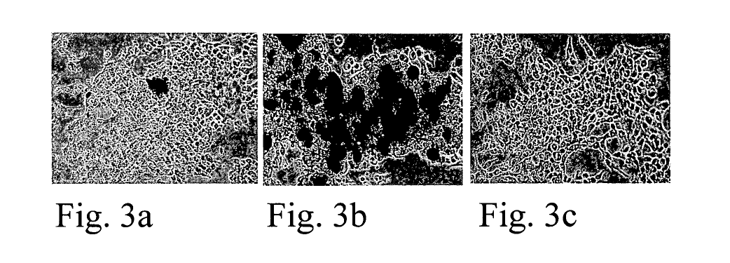

To test the responsiveness of the PSE-BC promoter to androgen, we infected LNCaP cells with Ad-PSE-BC/FLP and Ad-RSV/FRT2neo/lacZ and cultured them in the presence and absence of the androgen R1881. In the absence of R1881, following histochemical staining for β-galactosidase activity, very few cells stained blue (FIG. 3). In contract, in the presence of R1881, most cells stained blue, an indication that the PSE-BC promoter's response to the addition of androgen resulted in increased expression of Flp recombinase, which in turn resulted in more expression of the lacZ reporter gene.

### Example 2

### PSE-BC Promoter-Driven Flp Recombinase-Mediated DT-A Expression Specifically Kills PSA-Expressing Cells in Culture

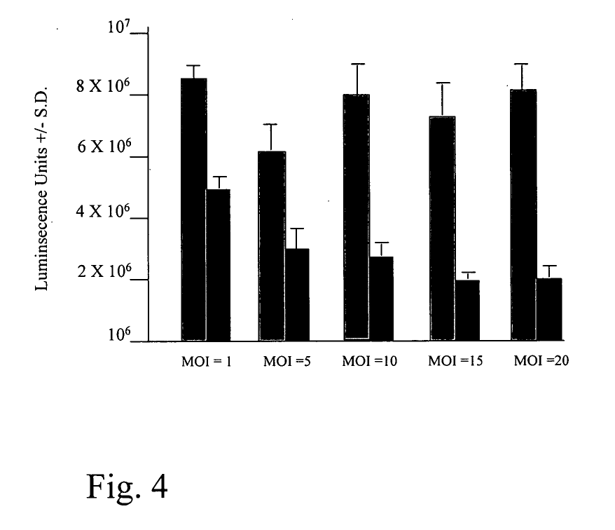

To test the ability of the PSE-BC promoter/Flp recombinase regulatory strategy to activate DT-A expression specifically in PSA-expressing cells in culture, we infected LNCaP cells, WI-38 fibroblasts, and human smooth muscle cells from coronary artery with Ad-PSE-BC/FLP and Ad-RSV/FRT2neo/DT-A. We then transfected cells with the plasmid, pCAG/luc. Twenty-four hours later, we assayed cell death indirectly by measuring luciferase enzyme activity. The level of luciferase activity was compared to control cells from each of the three lines that we infected with Ad-PST-BC/FLP and Ad-RSV/FRT2neo/LacZ. In experiments with PSA-expressing LNCaP cells, we also varied the amount of Ad-PSE-BC/FLP with which the cells were infected. Luciferase activity was 2-4 times lower in LNCaP cells infected with the DT-A virus as compared to cells infected with the LacZ control virus, the amount of reduction correlating with the Ad-PSE-BC/FLP dose (FIG. 4A). In contrast, we observed no difference in luciferase activity in WI-38 cells and smooth muscle cells (both non-PSA-expressing cells) that had been infected with the DT-A or the LacZ virus (FIG. 4B). These results demonstrate that the PSE-BC promoter shows specificity in its activity and that Flp recombinase-mediated recombination resulting in DT-A expression results in the death of PSA-expressing cells in culture.

### Example 3

### PSE-BC-driven Flp Recombinase-Mediated Gene Expression in Xenografts

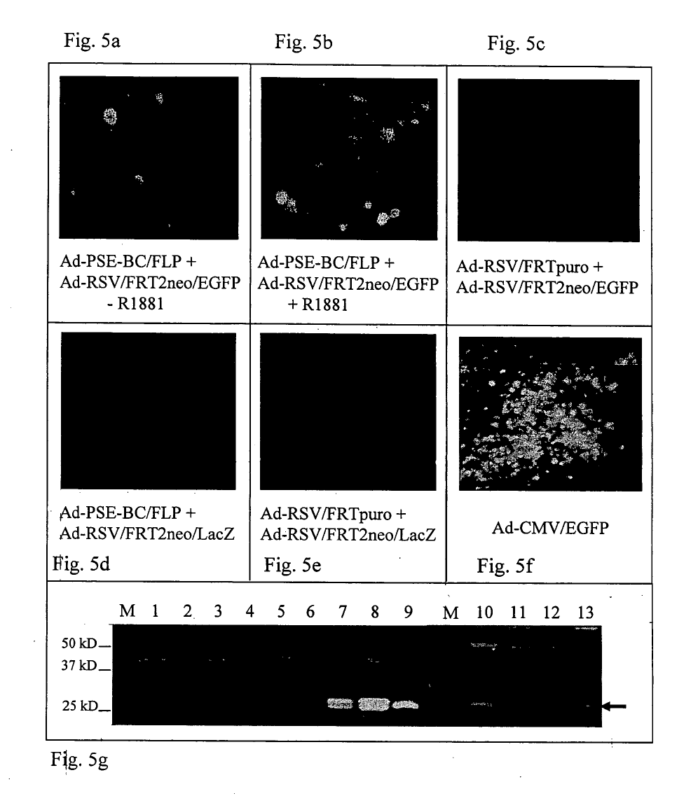

To test whether the PSE-BC promoter driven-regulatory system functions in vivo as it does in cells in culture, we inoculated nude mice subcutaneously with LNCaP cells to generate xenografts, and then administered Ad-PSE-BC/FLP and Ad-RSV/FRT2neo/EGFP intratumorally. In some cases, virus was administered in buffer containing 4 nM R1881. Forty-eight hours after injection of the viruses, tumors were excised from the host and observed as whole mounts under fluorescent light. While we observed green fluorescing cells throughout the tumors, there was an area in each tumor, presumably corresponding to the site of injection, where the frequency of fluorescing cells was higher than that of the surrounding area. Consistent with our observations in cells in culture, the addition of R1881 to the injection buffer resulted in an increase in the number of fluorescing cells in tumors (FIG. 5). Tumors injected with mixtures of control viruses, lacking either Flp recombinase or EGFP, or both, exhibited a few cells that fluoresced very weakly, presumably due to autofluorescence. In tumors injected with Ad-CMV/EGFP, the frequency of fluorescing cells was very high compared to the frequency in tumors inoculated with two viruses where Flp recombinase-mediated recombination is required for EGFP expression.

We prepared protein extracts of tumors 5- and 9-days after administration of different viral preparations (FIG. 5g) and subjected these extracts to Western blot analysis using an antibody to EGFP. We also included extracts of tumors injected with varying amounts of Ad-CMV/EGFP in our analysis. These samples served as positive controls and gauges with which to measure the sensitivity of the Western analysis. Five days post-injection of Ad-PSE-BC/FLP and Ad-RSV/FRT2neo/EGFP, we were unable to detect a 27 kD EGFP protein. However, we did detect EGFP in tumors 9-days post-injection. This observed increase in EGFP expression in tumors with the passage of time is consistent with our observations of whole tumors observed by fluorescent microscopy. It is not clear whether increased EGFP expression is the result of accumulated protein in the same number of expressing cells such that more cells are visibly fluorescing, or whether the increase is the result of clonal expansion of cells in which the DNA recombination event resulting in EGFP expression took place.

### Example 4

### Tumor Regression Following PSE-BC-Driven Flp Recombinase-Mediated DT-A Expression

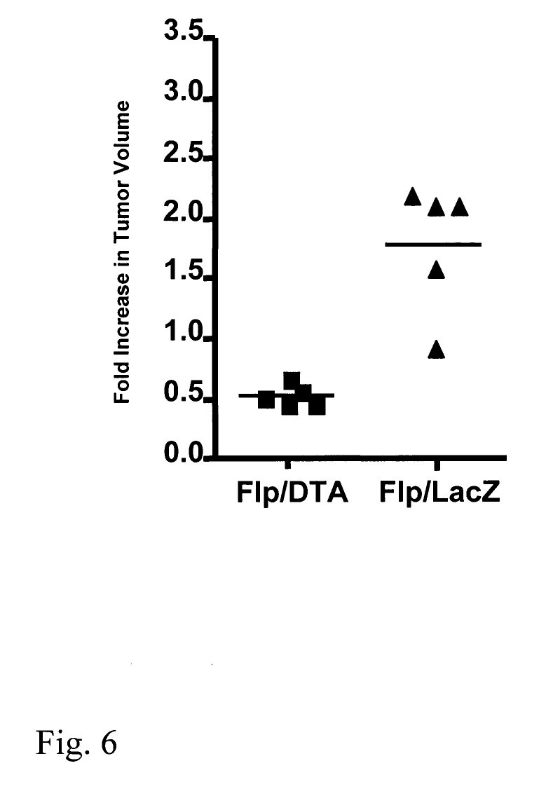

We next tested how effectively Flp recombinase-regulated expression of DT-A kills prostate tumor cells in xenografts. Following the development of xenografts deriving from LNCaP cells in nude mice, we used calipers to measure the size of each tumor and then administered a mixture of two viruses, either Ad-PSE-BC/FLP+Ad-RSV/FRT2neo/DT-A or Ad-PSE-BC/FLP+Ad-RSV/FRT2neo/LacZ, by intratumoral injection (1010 viral particles for each virus). Four days later, we administered the viruses a second time. Six days after the second injection, tumor size was again determined and mice were sacrificed. Following administration of Ad-PSE-BC/FLP and Ad-RSV/FRT2neo/DT-A, the tumors were half their original size (FIG. 6). In contrast, there was a 2-fold increase in the size of control tumors injected with Ad-PSE-BC/FLP and Ad-RSV/FRT2neo/LacZ. These results demonstrate that Flp recombinase-mediated expression of DT-A in PSA-expressing prostate tumor cells effectively kills the cells, resulting in tumor regression.

### Example 5

### Toxicity and Transfection Efficiency of Different Cationic Polymers

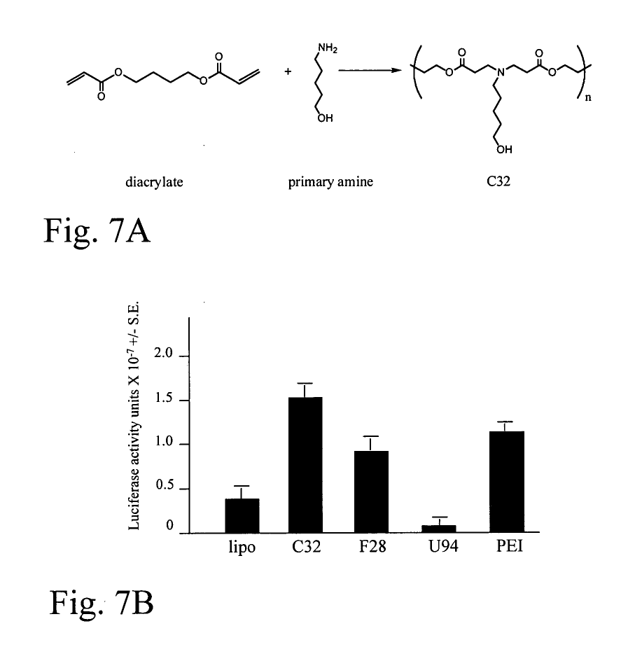

We evaluated toxicity associated with exposure of cells to nanoparticles containing different polymers as well as the efficiency with which different nanoparticle formulations transfected 293 cells in culture, using GFP and luciferase reporter gene constructs for this purpose. In these experiments, we also tested different polymer:DNA ratios (10:1, 20:1, 30:1, and 40:1) and different times of exposure (1 hr. and 3 hr.) to the nanoparticle-DNA complexes. Forty-eight hours after exposure of cells to nanoparticles, we assessed transfection efficiency either by observing them under fluorescent light (for GFP expression) or by measuring luciferase enzyme activity (for luciferase expression). Toxicity was assessed by visual inspection of the cells at various times after treatment with polymer. An increased number of non-adherent cells indicated increased toxicity. We determined that at a 30:1 polymer:DNA ratio, compared to other polymers tested, one polymer, C32, transfected cells with very high efficiency and was the least toxic to cells (FIG. 7). The transfection rate of C32 was comparable to that achieved with branched polyethylenimine (PEI), but C32 was less toxic than PEI.

### Example 6

### PSA Promoter/Enhance-Regulated Gene Expression Following Nanoparticle Delivery of DNA to Human Prostate Cancer Cells in Culture

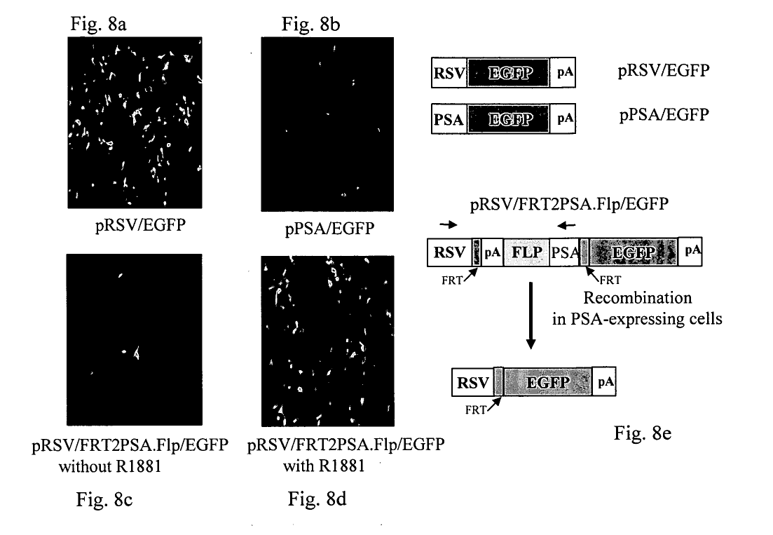

To test the ability of C32-nanoparticles to deliver DNA to human prostate cancer cells in culture, we incubated LNCaP cells for 1 hour with C32-DNA nanoparticles. The medium was changed and forty-eight hours later cells were observed using a fluorescent inverted microscope equipped with an FITC filter. Nearly all cells transfected with C32-pRSV/EGFP (Rous sarcoma virus promoter driving expression of humanized green fluorescent protein) expressed EGFP (FIG. 8a). The level of expression was variable; some cells fluoresced strongly, while the amount of fluorescence in other cells was much weaker. Fewer cells fluoresced after transfection with C32-pPSA/EFGP (a chimeric enhancer/promoter of human prostate specific antigen driving expression of EGFP), and the level of fluorescence was very low (FIG. 8b). When cells were transfected with C32-pRSV/FRT2PSA.Flp/EGFP, very few cells expressed EGFP, but those that did, fluoresced brightly (FIG. 8c). In order for EGFP to be expressed in these cells, Flp recombinase-mediated recombination must occur resulting in excision of the DNA between the two recombinase target sequences, thereby juxtaposing the RSV promoter with the EGFP coding sequence. We also transfected cells growing in medium containing 1 nM R1881, a synthetic androgen, with C32-pRSV/FRT2PSA.FLP/EGFP. R1881 has been shown to induce activity of the PSA enhancer/promoter, a consequence of multiple androgen receptor elements in this regulatory sequence (Wu, L., Matherly, J., Smallwood, A., Adams, J. Y., Billick, E., Belldegrun, A., and Carey, M., 2001, “Chimeric PSA enhancers exhibit augmented activity in prostate cancer gene therapy vectors,” Gene. Ther., 8:1416-1426). In the presence of R1881, the percentage of C32-pRSV/FRT2PSA.FLP/EGFP transfected cells expressing EGFP was nearly as high as the percentage of expressing cells after transfection with C32-pRSV/EGFP (compare FIGS. 8a and d). Many cells fluoresced very brightly suggesting that the RSV promoter was driving EGFP expression following Flp-mediated recombination. The results of this experiment indicate C32-nanoparticle-delivered DNA is expressed very efficiently in prostate cancer cells in vitro. The PSA enhancer/promoter is much weaker than the RSV promoter. Furthermore, Flp recombinase-mediated DNA recombination, driven by the PSA enhancer/promoter, occurred with high efficiency when activity of the PSA enhancer/promoter was hormonally induced.

### Example 7

### Nanoparticle-Delivered Diphtheria Toxin (DT-A) DNA Kills Cells in Culture

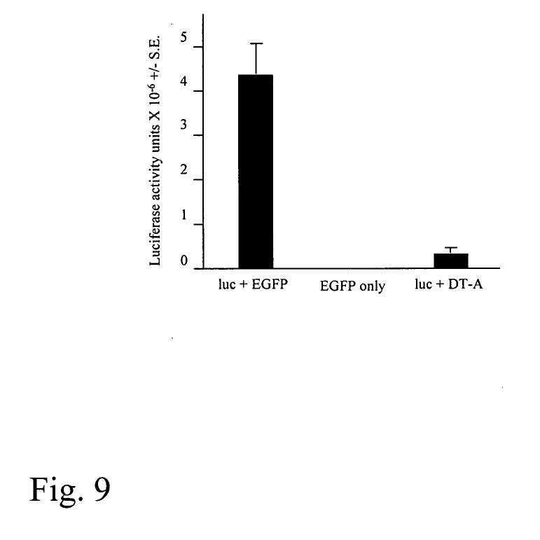

DT-A catalyzes the transfer of ADP-ribose from NAD to a modified histidine residue on elongation factor 2, thereby inhibiting protein synthesis (Collier, R. J., 1975, “Diphtheria toxin: mode of action and structure,” Bacteriol. Rev., 39:54-85). To test the ability of nanoparticle-delivered DT-A to kill prostate cancer cells, LNCaP cells were transfected with C32-pCAG/luc and with a second nanoparticle preparation, either C32-pRSV/FRT2PSA.FLP/EGFP or C32-pRSV/FRT2PSA.FLP/DT-A. We assayed cell death indirectly by measuring luciferase enzyme activity and determining whether activity was reduced in cells transfected with the DT-A construct as compared to cells transfected with the EGFP construct. Control cells were transfected with pRSV/FRT2PSA.FLP/EGFP only. As shown in FIG. 9, luciferase activity was reduced to near background level in cells transfected with the DT-A construct, while activity was very high in cells transfected with the EGFP construct. The results of this experiment demonstrate that nanoparticle delivery of a construct in which DT-A expression is regulated both transcriptionally and by DNA recombination effectively kills prostate cancer cells grown in culture.

### Example 8

### Expression of Nanoparticle-Delivered DNA in Xenografts

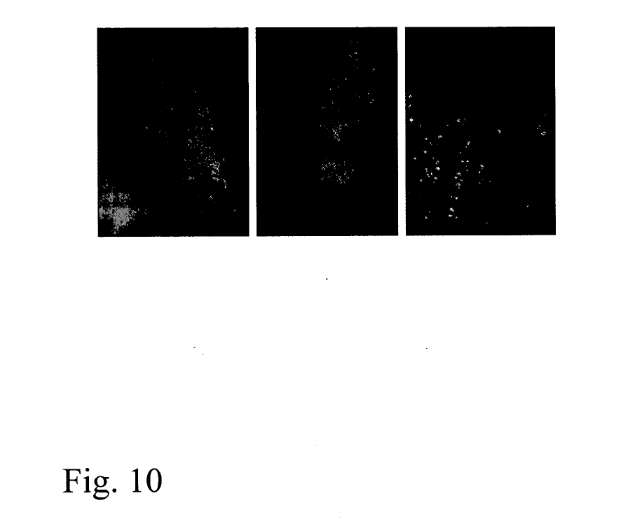

In addition to testing nanoparticle delivery of DNA to cells in culture, we have investigated the efficiency with which nanoparticle-delivered DNA is expressed in xenografts. LNCaP cells were mixed with Matrigel and inoculated subcutaneously into the flanks of nude mice to generate tumors. When tumor volumes were approximately 50 mm³, we injected C32-pCAG/EGFP nanoparticles intratumorally (50 μg DNA/injection, 30:1 polymer:DNA ratio). Forty-eight hours after injection, mice were euthanized, and tumors were removed and viewed by fluorescent microscopy. In every tumor we examined, we observed many GFP-expressing cells that were presumably at the area near the injection site (FIG. 10). Tumors injected with C32-pCAG/luc nanoparticles served as a negative control for fluorescence. No fluorescence was observed in these tumors.

### Example 9

### Tumor Regression Following Nanoparticle-Delivered DT-A DNA

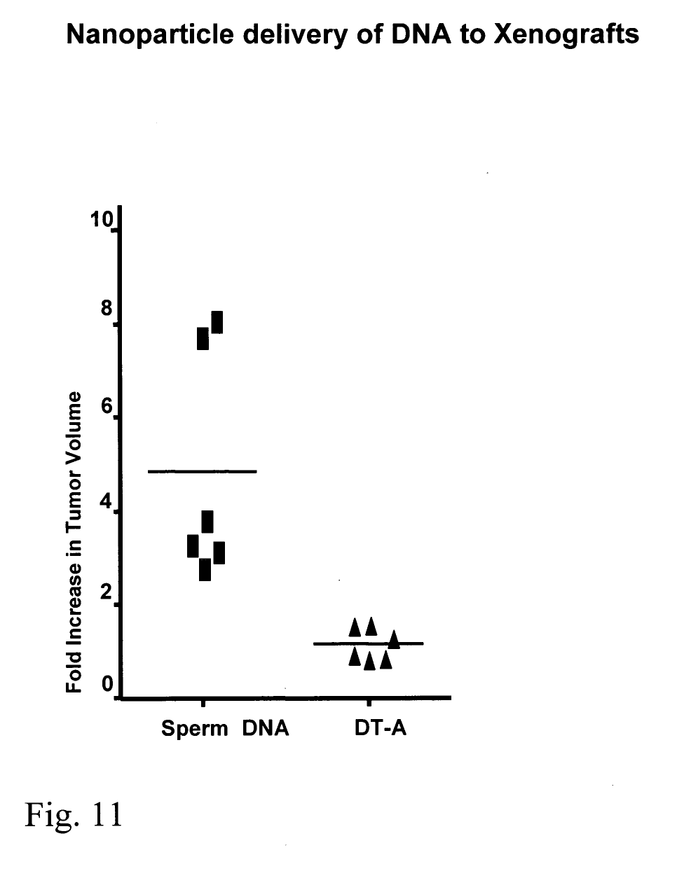

Having established that nanoparticles can effectively transfer DNA to xenografts, we wished to determine whether nanoparticle-delivered DT-A would kill tumor cells. As described above, LNCaP cells were injected into nude mice to generate tumors. When tumors attained a volume of approximately 50 mm³, we injected either C32-pRSV/FRT2PSA.FLP/DT-A or C32-salmon testes DNA intratumorally (50 μg DNA/injection, 30:1 polymer:DNA ratio). We administered nanoparticles to tumors two more times, three and ten days after the first injection. We used calipers to measure tumor size three and eight days after the first injection, and one day after the final injection (day 11), at which time mice were euthanized. On day 11, all tumors injected with C32-pRSV/FRT2PSA.FLP/DT-A were smaller than tumors injected with salmon testes DNA (FIG. 11). C32-delivery of the DT-A encoding DNA to xenografts resulted in a 5-fold reduction in tumor volume as compared to control tumors.

### Example 10

### PSE-BC-Driven Flp Recombinase-Mediated Gene Expression in Autochthomous

Intratumoral injection (Ad-PSE-BC/FLP+Ad-RSV/FRT2neo/EGFP) and systemic injection (Ad-CAG/luc and Ad-PSA/FLP+Ad-RSV/FRT2neo/luc) in mice were tested.

Those skilled in the art will recognize, or be able to ascertain using no more than routine experimentation, many equivalents to the specific embodiments of the invention described herein. Such equivalents are intended to be encompassed by the following claims.

The disclosures of all scientific and patent publications and references disclosed herein are incorporated herein by reference in their entirety.

1. A composition comprising:
a first nucleic acid comprising a first promoter operably joined to a sequence encoding a recombinase,
a second nucleic acid comprising a second promoter, a sequence encoding a toxin, and at least one target sequence that is recognized by the recombinase,
wherein the second promoter and the sequence encoding the toxin are arranged such they are only operably joined if the recombinase acts on the at least one target sequence, and wherein the first promoter is a tissue specific promoter that is selectively active in non-vital tissue.

2. The composition of claim 1, wherein the first promoter is selectively active in a non-vital organ.

3. The composition of claim 2, wherein the non-vital organ is breast, thyroid, ovary, or testes.

4. The composition of claim 2, wherein the non-vital organ is a prostate.

5. The composition of claim 1, wherein the first promoter is a modified promoter.

6. The composition of claim 1, wherein the first nucleic acid further comprises an enhancer element.

7. The composition of claim 1, wherein the first promoter is a modified prostate specific promoter.

8. The composition of claim 7, wherein the modified prostate specific promoter is a modified PSA promoter.

9. The composition of claim 8, wherein the modified PSA promoter is PSA-BC.

10. The composition of claim 1, wherein the second promoter is a tissue-specific promoter.

11. The composition of claim 1, wherein the second promoter is not tissue-specific.

12. The composition of claim 1, wherein the second promoter is an xenogeneic promoter.

13. The composition of claim 12, wherein the xenogeneic promoter is a mammalian promoter.

14. The composition of claim 12, wherein the xenogeneic promoter is a viral promoter.

15. The composition of claim 1, wherein the toxin is a protein.

16. The composition of claim 1, wherein the toxin is an enzyme.

17. The composition of claim 15, wherein the protein is a modified natural protein.

18. The composition of claim 15, wherein the protein is the A chain of diphtheria toxin.

19. The composition of claim 1, wherein the first and second nucleic acids are covalently linked.

20. The composition of claim 19, wherein the first and second nucleic acids are comprised by a single linear nucleic acid.

21. The composition of claim 19, wherein the first and second nucleic acids are comprised by a single circular nucleic acid.

22. A nucleic acid delivery preparation comprising the nucleic acid of claim 1 and a cationic polymer.

23. A nucleic acid delivery preparation comprising a cationic polymer and a nucleic acid composition comprising:
a first nucleic acid comprising a first promoter operably joined to a sequence encoding a recombinase,
a second nucleic acid comprising a second promoter, a sequence encoding a toxin, and at least one target sequence that is recognized by the recombinase,
wherein the second promoter and the sequence encoding the toxin are arranged such they are only operably joined if the recombinase acts on the at least one target sequence.

24. A method of targeting a toxin to a non-vital cell type, the method comprising contacting a cell with a composition of claim 1, wherein the first promoter is selectively active in the cell.

25. The method of claim 24, wherein the non-vital cell type is a prostate epithelial cell.

26. A method of targeting a toxin to a specific cell type, the method comprising contacting a cell of a specific cell type with the nucleic acid delivery preparation of claim 23, wherein the first promoter is selectively active in the specific cell type.

27. A method of targeting a toxin to a diseased cell type, the method comprising administering to a subject having a disease a nucleic acid composition comprising:
a first nucleic acid comprising a first promoter operably joined to a sequence encoding a recombinase,
a second nucleic acid comprising a second promoter, a sequence encoding a toxin, and at least one target sequence that is recognized by the recombinase,
wherein the second promoter and the sequence encoding the toxin are arranged such they are only operably joined if the recombinase acts on the at least one target sequence,
wherein the first promoter is selectively active in a population of cells comprising diseased cells, and
wherein the nucleic acid composition is administered non-virally.

28. The method of claim 27, wherein the nucleic acid composition is a plasmid.

29. The method of claim 27, wherein the nucleic acid composition is administered in combination with a cationic polymer.

30. The method of claim 27, wherein the first promoter is selectively active in cancer cells.

31. The method of claim 27, wherein the first promoter is tissue specific.

32. The method of claim 27, wherein the disease is cancer.

33. A method of treating cancer comprising, administering a composition of claim 1 to a subject that has cancer.

34. A method of treating cancer comprising, administering a nucleic acid delivery preparation of claim 23 to a subject that has cancer.

35-38. (canceled)

39. The composition of claim 1, wherein the recombinase is Flp recombinase.

40. A nucleic acid delivery preparation of claim 22, wherein the cationic polymer is a poly(β-aminoester).

41. The nucleic acid delivery preparation of claim 40, wherein the cationic polymer is C32.
